SEQUENCE LISTING (3 sequences)

SEQ ID NO: 1 - DNA, 2119 bp, Homo sapiens
ggatccttctaagctcattcaagaattttgggctttaactatttcctttgatttaacctggtaccaggtgccaactttagataatagggatatctaattacttctaaattcctcagataaggggcctgcttgatggtcaccaggtgatctgtgctctccttaagagggaataagacctagcgttggcagagttctgtagggtgactatagttaacagtaatctgttgtatattttaaaatgttattattgaagagagtaactggaatgttcccagtataaagacaaatgtttaaggtgatagagatctcatttaccctgatttaatcattacacattatatgaaagtatcaaaataccacatgtacccagaaaacacatacgtctcttacatatcaataaatacaacttgagattatgatgtaaatacatctgaccaacttggtacttattagacttatgtgcgcagcactgctctagtcctgtgggtgcagcagcatcaggatcgttaaagaaaacaaacaatgctgagaaaaaaactcacacccctgagacatccgggtgtgaataaatgcggcagagtcgcccgagatcgggagaccaggcgtgggggagaggtccgggaggcctggaccagagtcctaacagaccagaggcgaaacgggaaggcgcgccagaaaaggaacaacgcaaagggagcaggcgtgcacggagcgcgaactaaggaacccctctgacaaccccagtccctcggcagttccagagaccggctcctcacggagggtggcggtagagactgttaagccccgcgggcgccggggcaggccggactgtgccattcgtggggggtaccatgtgggaccgagccgcctcacccagggctgtccagctagaaactccccggtgccacccccgcctcagtccgaggtagactcggccggacgtgacgcagcgtgaggccaaggcggcgtgagtctgcgcagtgtggggctgagggaggccggacggcgcgcgtgcgtgctggcgtgcgttcactttcagcctggtgtggggtgagtggtacccaacgggccggggcgccgcgtccgcaggaagaggcgcggggtgcaggtcagcgccagcgggggcgcggcgcatgtgtgggccgtggcgctgggcggcgtgggggtgctggacggtgtccctgtgctggacggtgtcccgctggctcagaaccggcgcggggcctgggtcggggccgccctcgcttccggcctcccagtcgggccctgtcgctggcgttggatttgactgaccgccagcgtggtggcaacgctgaagcgtccagaatcttctgcctaacctctcgccggcatggaactggctagccgttttattaaactctgttttgcgtggacggtaaaccctccagataatctgtaaataggttaaaaaaaattcggaacctcgttgagctgctgtcgttggcagtgagaactccgcgcagagagacagatgtagttgggttgacttcagtgaggggatttccatctttctcagtcattaaaaaaagtgttcagacatttaacactgttgacccccacacacaattttttagtacagttataactaagaaaacaaaaatcccctccaaaaaattacaagttaattgcgaaagaccacatttaaatttttgcccatgaaattcagtttagtcgtttctctgaaacagtgcttcaaaaaagactgtttccccgcattgtgtgaaatgcaggagacccacgtacttgtatttttaaaaaacccatttgcaacatactattaaagttggatttaagagaacatggtagaagaaaatctaagcaatactacaccttttagcaccctcattatgttttcatctcagagcaattaaaactgctatacaaatcaacgttaagataactaaactgctgcttttttcgtattcagttgtctatgaaaaccgtttccctaggaagtacttactctgcttgaaaatgctcctaaactttaaattttggggtatctcagggttgcaatgaaagttttttgaaatctttttttttttttttttttaaggcttgtaaacatataacataaaaatggcttccaaaagagctc

SEQ ID NO: 2 - DNA, 36 bp, Artificial Sequence
  Description of Artificial Sequence Synthetic primer
ggtggtctcgagggatccttctaagctcattcaaga

SEQ ID NO: 3 - DNA, 31 bp, Artificial Sequence
  Description of Artificial Sequence Synthetic primer
ggaggaaagcttttgggtaccactcacccca

(19) United States
(12) Patent Application Publication
(10) Pub. No.: US 2010/0047776 A1
(43) Pub. Date: Feb. 25, 2010

(54) Cell-based screening assays for compounds that regulate the expresion of a tumor marker dj-1

(75) Inventor: Jin Xu, Wellesley, MA (US)

Correspondence Address: EDWARDS ANGELL PALMER & DODGE LLP, P.O. BOX 55874, BOSTON, MA, 02205, US

(73) Assignee: Caritas St. Elizabeth Medical Center of Boston Inc., Boston, MA (US)

(21) Appl. No.: 11/920,091

(22) PCT Filed: May 8, 2006
(86) PCT No.: PCT/US06/17850

§ 371 Date: Jan. 9, 2009

Related U.S. Application Data
(60) Provisional application No. 60/679,764, filed on May 10, 2005.

(51) Int. Cl. C12Q 1/68; C12N 15/63; C12N 5/10

(52) U.S. Cl. 435/6; 435/320.1; 435/366

(57) ABSTRACT

The present invention generally provides compositions and methods that are useful for the treatment or prevention of a neoplasm, and screening methods that can be used to detect compounds that modulate the activity of a DJ-I gene.

### CROSS-REFERENCE TO RELATED APPLICATION

This application claims the benefit of U.S. Provisional Application No. 60/679,764, which was filed on May 10, 2006 the entire disclosure of which is hereby incorporated in its entirety.

### FIELD OF THE INVENTION

The invention generally relates to compositions and methods capable of preventing, treating, or delaying the onset of a neoplasm. In one embodiment, the invention provides specialized cells and screening paradigms that can be used to detect such compounds, particularly in high- or ultra-high throughput screening formats. Practice of the invention has a wide spectrum of useful applications including identifying compounds that alter the transcription of genes required for neoplastic cell survival, proliferation or metastasis.

### BACKGROUND OF THE INVENTION

Neoplasia results when a cell acquires a genetic mutation that allows it to evade the normal regulatory mechanisms controlling cell growth and proliferation. Neoplasia is the second leading cause of death in the United States. Nearly half of all men and more than one third of all women in the United States will develop a neoplasia during their lifetimes. Internationally, there were 6.7 million deaths related to neoplasia between 2000 and 2005. Because existing methods of treating neoplasia are inadequate, new therapeutics and screening methods to identify such therapeutics are required.

### SUMMARY OF THE INVENTION

The present invention generally provides compound screening methods that can be used to detect compounds that specifically modulate the activity of a DJ-1 gene. The invention has a wide spectrum of useful applications, including identifying compounds that can be used to alter the transcription of genes required for neoplastic cell survival, proliferation or metastasis.

The DJ-1 protein has been shown to promote the survival of neoplastic cells. As will be described in more detail below, the methods and compositions of the invention are useful for the identification of compounds that modulate DJ-1 expression. Regulating transcription of a DJ-1 gene is an important new way of preventing, treating, delaying the onset of, or reducing symptoms associated with a neoplasm. By “neoplasm” is meant a disease or disorder caused by inappropriately high levels of cell division, inappropriately low levels of apoptosis, or both. Neoplastic diseases include, for example, lymphoma, leukemia, melanoma, thyroid cancer, ovarian cancer, breast cancer, pancreatic cancer, and lung cancer. It is thus an object of the present invention to provide in vitro and in vivo screening tools that can be used to detect and identify compounds that can be used as anti-neoplastic therapeutics.

In general, the invention features a method for detecting a candidate compound (e.g., a compound present in a chemical library) that can stabilize the growth, reduce the survival, or inhibit the invasiveness of a neoplastic cell. The method involves (a) contacting a recombinant cell containing a DJ-1 expression vector with at least one candidate compound; and (b) detecting an alteration in expression of DJ-1 in the contacted cell relative to a reference (e.g., expression in an uncontacted control cell). In one embodiment, DJ-1 expression is detected using a DJ-1 expression vector that contains between about 500 and 2000 base pairs (e.g., 500, 750, 1000, 1250, 1500, 2000) upstream of a DJ-1 transcription start site operably linked to a detectable (e.g., luciferase) reporter. Desirably, a compound selected using the methods of the invention reduces the expression of a DJ-1 nucleic acid molecule or protein by at least 10%, 25% or 50%, or by as much as 75%, 85%, 90%, or 95%.

In one embodiment, reporter expression is detected using a fluorometric assay, where the fluorescent signal is detected using a fluorescence microscope, fluorometer, fluorescence microplate reader, fluorescence activated cell sorter or flow cytometer. Such methods may be conducted in an automated, semi-automated, or manual format. Preferably, a high- or ultra-high throughput screening assay is used. Such assays typically involve any one or more of the following components: (1) microtiter plates, (2) fluorometer, fluorescence microplate reader, flow cytometer or luminometer. In general, high or ultra-high throughput screening is facilitated by the use of a computer. The computer may be interfaced with the fluorometer, fluorescence microplate reader, flow cytometer or luminometer to receive input from the assay and provide output data to a user. The output data is stored and optionally manipulated by the computer or released to the user in real-time. In another embodiment, reporter expression is detected using an immunological assay, an enzymatic assay, or a radioimmunoassay.

In another embodiment, the method further involves selecting the candidate compound providing the alteration in reporter expression. If desired, the selected compound is tested in a cell growth assay (e.g., an assay that detects DNA replication), where growth stabilization or growth inhibition identifies the compound as one that stabilizes or inhibits the growth of a neoplastic cell. In one embodiment, the method further involves testing the selected compound in a cell survival assay, where a reduction in cell survival identifies the compound as one that reduces the survival of a neoplastic cell. Cell survival is detected, for example, by detecting apoptosis (e.g., trypan blue or TUNEL assay). In another embodiment, the method further involves testing the selected compound in an assay that detects metastatic potential, where a compound that reduces metastatic potential is identified as inhibiting the invasiveness of a neoplastic cell.

In some embodiments, it may be desirable to test selected compounds in an animal, such as a rodent (e.g., a mouse or rat) that optionally contains a neoplasm. Preferably, compounds selected according to the methods of the invention reduce the growth, proliferation, or severity of the neoplasm by at least 10%, 25%, or 50%, or by as much as 75%, 85%, or 95% when compared to a control. In other embodiments, compounds are tested in a worm (e.g., C. elegans) or a fruit fly (e.g., Drosophila) that expresses a recombinant DJ-1 nucleic acid molecule or an endogenous DJ-1 nucleic acid molecule.

As will become apparent from the following disclosure, the above-mentioned screen is flexible and can be adapted to suit an intended screening paradigm. Thus in one embodiment, the foregoing screen is combined with one or more in vivo assays disclosed herein to further select useful compounds. Preferred in vivo assays use an acceptable animal model of neoplastic disease (e.g., rodent, rabbit, primate, insect, nematode models). Thus, in one embodiment, the method further includes testing the compound in an acceptable animal model. Preferred methods “pre-screen” compounds for suitable activity in one or more of the in vitro screens of the invention.

Practice of the invention is compatible with testing compounds either alone as a sole active agent or in combination with other active compounds such as those currently in use to treat certain neoplasms, such as tomoxifin, cisplatin, vincristin, and flutamide.

Significantly, the use of multiple detection formats (i.e., a combination of in vitro assay, a combination of in vivo assays, or a combination of both in vitro and in vivo assays) with a single candidate compound can extend the selectivity and sensitivity of the detection desired.

Such broad spectrum testing provides advantages, such as increasing the chances of detecting compounds with therapeutic activity. This is especially useful when large compound batches are to be analyzed. For instance, and as disclosed below, such candidate compounds can be derived from available compound libraries or can be made using standard synthetic methods including combinatorial-type chemistry manipulations and then tested in accord with the invention.

Notwithstanding the ability to combine various assays of the invention, it will often be useful to focus initial efforts on testing large numbers of compounds in vitro, for example, in a high-throughput assay. In these embodiments, it will often be useful to make recombinant cell lines that include expression vectors that can be used in accord with the invention to detect compound induced changes in DJ-1 gene activity. Accordingly, and in another aspect, the invention provides a suitable expression vector that includes a DJ-1 promoter sequence operably linked to at least one reporter sequence of the vector. The precise promoter sequence used in each vector will vary, depending on the level of assay sensitivity and the efficiency of certain recombinant manipulations, such as cell transformation. In most instances, the vectors will include less than about 4000 base pairs “upstream” of the transcription start site of the DJ-1 gene. In various embodiments, as little as 100, 250, 500 or 1000 nucleotides of upstream sequence is used, or as much as 1500, 2000, 2500, 3000, or 3500 is used.

In some embodiments, additional DJ-1 sequence is inserted into the expression vectors of the invention. Such regulatory sequence, if needed at all, may improve assay sensitivity and selectivity. In these embodiments, the expression vector will desirably include sequence “downstream” of the transcription start site of each of the DJ-1 gene. In some embodiments, this downstream sequence is operably linked to the promoter sequence. Preferably, such “downstream” sequence information includes less than about 500 base pairs of downstream sequence (e.g., 25, 50, 75, 100, 200, 250, 300, or 400 nucleotides of regulatory sequence).

In another aspect, the invention provides recombinant cells and recombinant cell lines that include at least one of the expression vectors disclosed herein. Such cells are derived from primary, secondary, or tertiary sources (cells, tissue) as needed to suit an intended invention objective. Suitable cells lines can be immortalized. As discussed below, the invention is compatible with a wide variety of cells and cell lines, although for many applications cells derived from neuronal, breast, lung, testis, or prostate tissue sources will often be useful. Such cells and cell lines can, in some embodiments, maintain the expression vector transiently. However, in other embodiments, more long term and stable maintenance of the expression vector by the cells will be desirable. Methods of obtaining transient or stable recombinant cell lines are known in the art (Transformation and transfection methods are described, e.g., in Ausubel et al. in Current Protocols in Molecular Biology, John Wiley & Sons, New York (1989); expression vehicles may be chosen from those provided, e.g., in Cloning Vectors: A Laboratory Manual (P. H. Pouwels et al., 1985, Supp. 1987). and are described herein.

Further provided by the invention is a method for producing the recombinant cell lines provided herein. In one embodiment, the method involves contacting a cell with the expression vector under conditions conducive to introducing the vector into the cell; and transforming the cell to make a recombinant cell line. More specific methods are discussed below.

In another aspect, the invention provides a kit that includes at least one of: the recombinant cell lines of the invention; and at least one of the expression vectors.

Other features and advantages of the invention will be apparent from the detailed description, and from the claims.

### BRIEF DESCRIPTION OF THE DRAWINGS

FIG. 1 is a schematic drawing showing the −1000DJ-Luc expression vector. The human DJ-1 promoter was linked to a luciferase reporter gene as described in methods.

FIG. 2 is a graph showing activation of the human DJ-1 promoter by H₂O₂.

FIG. 3 is a Western blot showing that phosphorylation of Akt is increased in cells over-expressing wild-type DJ-1.

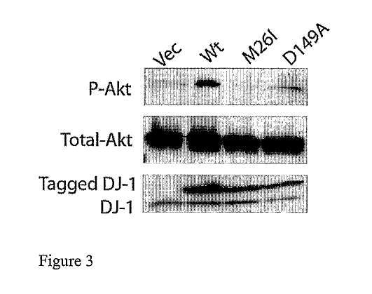

### DETAILED DESCRIPTION OF THE INVENTION

The present invention provides compositions and methods that can be used to detect compounds that modulate the activity of a DJ-1 gene. The DJ-1 protein has been shown to promote the survival of neoplastic cells, and is over-expressed in a number of cancers, such as breast and lung cancer. As will be described in more detail below, the methods and compositions of the invention are useful for the identification of compounds that modulate DJ-1 expression. In one embodiment, compounds detected by the invention decrease the activity of DJ-1, typically by decreasing promoter function. The invention has a wide spectrum of useful applications, including identifying compounds that can be used to prevent, treat, defer the onset of or help alleviate symptoms associated with certain neoplasms. In another embodiment, selected compounds fail to cross the blood brain barrier, and so are limited in distribution to tissues outside of the central nervous system.

Illustrative neoplasms for which the invention can be used to detect new therapeutic compounds (or confirm activity of existing compounds) include, but are not limited to leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). In one embodiment, screening methods of the invention identify compositions that are useful for treating breast or lung cancer.

The present invention further provides methods of treating a neoplastic disease and/or disorders or symptoms thereof which comprise administering a therapeutically effective amount of a pharmaceutical composition identified according to a method disclosed herein to a subject (e.g., a mammal such as a human). Thus, one embodiment is a method of treating a subject suffering from or susceptible to a neoplastic disease or disorder or symptom thereof. The method includes the step of administering to the mammal a therapeutic amount of an amount of a compound herein (e.g., a compound that reduces DJ-1 polypeptide or nucleic acid molecule expression) sufficient to treat the disease or disorder or symptom thereof, under conditions such that the disease or disorder is treated.

The methods herein include administering to the subject (including a subject identified as in need of such treatment) an effective amount of a compound identified according to a method described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which DJ-1 overexpression may be implicated.

As discussed herein, the invention provides a variety of expression vectors that can be used to make useful recombinant cell lines. Such cell lines can be used in accord with the invention to screen for compounds that can be used therapeutically to help prevent, treat, reduce the severity of, or prolong the onset of a neoplasm.

Thus in one invention aspect, there is provided an expression vector that includes a DJ-1 promoter sequence operably linked to at least one reporter sequence. By “promoter sequence” and related phrases such as “promoter” including plural forms, is meant a DNA sequence capable of controlling the expression of an oligonucleotide sequence to which it is operably linked. The expression of the oligonucleotide may be as a primary RNA transcript or as an mRNA. A promoter is typically located 5′ (i.e., upstream) of an oligonucleotide sequence whose transcription it controls, and the promoter usually provides a site for specific binding by RNA polymerase and for the initiation of transcription. The term “promoter activity” when made in reference to a nucleic acid sequence (typically a promoter) refers to the ability of the nucleic acid sequence to initiate transcription of an oligonucleotide sequence into mRNA. Preferred promoter sequences are discussed in more detail below.

DNA regions are referred to as “operably linked” when they are functionally related to each other. For example: a promoter sequence is operably linked to a coding sequence if it controls the transcription of the sequence; a ribosome-binding site is operably linked to a coding sequence if it is positioned so as to permit translation. Generally, operably linked means contiguous and, in the case of leader sequences, contiguous and in reading frame.

### Making Expression Vectors

A. Promoter Sequence Information

### DJ-1

The sequence of the human and mouse DJ-1 genes has been reported. See Taira, T. et al. (2001) Gene 263: 285. In particular, the human DJ-1 promoter, gene and 5-UTR have been disclosed by GenBank as Accession Number AB045294. See the National Center for Biotechnology Information (NCBI)-Genetic Sequence Data Bank (Genbank), National Library of Medicine, 38A, 8N05, Rockville Pike, Bethesda, Md. 20894. See Benson, D. A. et al. (1997) Nucl. Acids. Res. 25: 1 for a description of this sequence.

The sequence disclosed by GenBank as accession no. AB045294 is shown below in Table 1.

TABLE I

| | |
|---|---|
| 1 | ggatccttct aagctcattc aagaattttg ggctttaact atttcctttg atttaacctg |
| 61 | gtaccaggtg ccaactttag ataataggga tatctaatta cttctaaatt cctcagataa |
| 121 | ggggcctgct tgatggtcac caggtgatct gtgctctcct taagagggaa taagacctag |
| 181 | cgttggcaga gttctgtagg gtgactatag ttaacagtaa tctgttgtat attttaaaat |
| 241 | gttattattg aagagagtaa ctggaatgtt cccagtataa agacaaatgt ttaaggtgat |
| 301 | agagatctca tttaccctga tttaatcatt acacattata tgaaagtatc aaaataccac |
| 361 | atgtacccag aaaacacata cgtctcttac atatcaataa atacaacttg agattatgat |
| 421 | gtaaatacat ctgaccaact tggtacttat tagacttatg tgcgcagcac tgctctagtc |
| 481 | ctgtgggtgc agcagcatca ggatcgttaa agaaaacaaa caatgctgag aaaaaaactc |
| 541 | acacccctga gacatccggg tgtgaataaa tgcggcagag tcgcccgaga tcgggagacc |
| 601 | aggcgtgggg gagaggtccg ggaggcctgg accagagtcc taacagacca gaggcgaaac |
| 661 | gggaaggcgc gccagaaaag gaacaacgca aagggagcag gcgtgcacgg agcgcgaact |
| 721 | aaggaacccc tctgacaacc ccagtccctc ggcagttcca gagaccggct cctcacggag |
| 781 | ggtggcggta gagactgtta agccccgcgg gcgccggggc aggccggact gtgccattcg |
| 841 | tggggggtac catgtgggac cgagccgcct cacccagggc tgtccagcta gaaactcccc |
| 901 | ggtgccaccc ccgcctcagt ccgaggtaga ctcggccgga cgtgacgcag cgtgaggcca |
| 961 | aggcggcgtg agtctgcgca gtgtggggct gagggaggcc ggacggcgcg cgtgcgtgct |
| 1021 | ggcgtgcgtt cactttcagc ctggtgtggg gtgagtggta cccaacgggc cggggcgccg |
| 1081 | cgtccgcagg aagaggcgcg gggtgcaggt cagcgccagc gggggcgcgg cgcatgtgtg |
| 1141 | ggccgtggcg ctgggcggcg tgggggtgct ggacggtgtc cctgtgctgg acggtgtccc |
| 1201 | gctggctcag aaccggcgcg gggcctgggt cggggccgcc ctcgcttccg gcctcccagt |
| 1261 | cgggccctgt cgctggcgtt ggatttgact gaccgacagc gtggtggcaa cgctgaagcg |
| 1321 | tccagaatct tctgcctaac ctctcgccgg catggaactg gctagccgtt ttattaaact |
| 1381 | ctgttttgcg tggacggtaa accctacaga taatctgtaa ataggttaaa aaaaattcgg |
| 1441 | aacctcgttg agctgctgtc gttggcagtg agaactccgc gcagagagac agatgtagtt |
| 1501 | gggttgactt cagtgagggg atttccatct ttctcagtca ttaaaaaaag tgttcagaca |
| 1561 | tttaacactg ttgaccccca cacacaattt tttagtacag ttataactaa gaaaacaaaa |
| 1621 | atcccctcca aaaaattaca agttaattgc gaaagaccac atttaaattt ttgcccatga |
| 1681 | aattcagttt agtcgtttct ctgaaacagt gcttcaaaaa agactgtttc cccgcattgt |
| 1741 | gtgaaatgca ggagacccac gtacttgtat ttttaaaaaa cccatttgca acatactatt |
| 1801 | aaagttggat ttaagagaac atggtagaag aaaatctaag caatactaca ccttttagca |
| 1861 | ccctcattat gttttcatct cagagcaatt aaaactgcta tacaaatcaa cgttaagata |
| 1921 | actaaactgc tgcttttttc gtattcagtt gtctatgaaa accgtttccc taggaagtac |
| 1981 | ttactctgct tgaaaatgct cctaaacttt aaattttggg gtatctcagg gttgcaatga |
| 2041 | aagttttttg aaatcttttt tttttttttt ttttaaggct tgtaaacata taacataaaa |
| 2101 | atggcttcca aaagagctc |

According to the sequence information provided in Table I, the transcription initiation site (start site) of the human DJ-1 gene is at nucleotide position 1016. Accordingly, the sequence provides about 1000 base pairs 5′ to (upstream) in relation to the transcription start site. It further provides about 1100 base pairs 3′ to (downstream) of the transcription start site.

Thus by the term “upstream” is meant in the 5′ direction to a particular reference point which in some instances will be a gene transcription start site (i.e., the nucleotide position that begins the primary RNA transcript). Similarly, “downstream” means in the 3′ direction to the particular reference point.

DJ-1 is a breast cancer marker that is overexpressed in breast and lung cancers. By identifying compounds that can modulate DJ-1 gene expression, it is possible to identify agents that alter various activities controlled by DJ-1 in tumorigenesis. Recombinant cells of the invention serve as an efficient and reliable resource to generate leads for drug discovery.

Preferred “functional portions” of a given promoter sequence are those that are capable of driving the transcription of an operably linked detectable reporter sequence encoded by the expression vector. In one embodiment, each of the DJ-1 promoter sequence includes about 2500 base pairs or less upstream of the respective DJ-1 transcription start site, for instance, about 1500 or 1000 base pairs upstream of the DJ-1 transcription start site or less. Additionally, suitable functional portions of such promoter sequences include those constructs having about 500 base pairs upstream of the respective DJ-1 transcription start site e.g., about 100 or about 50 or less base pairs upstream of the DJ-1 transcription start site.

In many instances, the functional portion of the DJ-1 promoter sequences will span between about 200 base pairs to about 2500 base pairs upstream of the respective transcription start site, such as 250 to about 1500 base pairs.

A suitably functional portion of the DJ-1 promoters is, in one embodiment, a component of the presently claimed expression vectors. The term “vector” means any nucleic acid sequence of interest capable of being incorporated into a host cell and resulting in the expression of a nucleic acid sequence of interest. Vectors can include, for example, linear nucleic acid sequences, plasmids, cosmids, phagemids, and extrachromosomal DNA. In many embodiments, the vector will be a plasmid or related sequence that is replicable in bacteria. Specifically, the vector can be a recombinant DNA. Also used herein, the term “expression” or “gene expression”, is meant to refer to the production of the detectable reporter sequence including transcription of its DNA and translation of its RNA transcript. Suitable expression vectors in accord with the invention will include, for instance, a “cloning site” that will be understood to include at least one restriction endonuclease site. Typically, multiple different restriction endonuclease sites (e.g., a polylinker) are contained within the nucleic acid.

Thus, the term “expression vector” including plural forms, means preferred vectors that include a DJ-1 gene promoter (or functional portion thereof) operably linked to a nucleic acid sequence that encodes a detectable reporter. Transcriptional activation of the promoter sequence is monitored by detecting expression of the reporter sequence, which typically encodes a detectable amino acid sequence.

Conventional procedures were also used to make vector DNA, cleave DNA with restriction enzymes, ligate and purify DNA, transform or transfect host cells, culture the host cells, and isolate and purify proteins and polypeptides. See generally Sambrook et al., Molecular Cloning (2d ed. 1989), and Ausubel et al. in Current Protocols in Molecular Biology, John Wiley & Sons, New York (1989),

Additional sequences encompassed by the invention include those sequences, such as primers and probes, that are capable of hybridizing under “stringent” conditions with one of the sequences provided in Table 1 as SEQ ID No.1. In one example, a stringent condition is a high stringency condition. Such conditions are known in the field and include, but are not limited to, hybridization conditions involving a wash at about 65° C. in 0.1×SSC (or 0.1×SSPE). See, for example, Sambrook et al., supra for more information relating to performing a high stringency hybridization.

Additional promoter sequences encompassed by this disclosure includes a promoter sequence or functional portion thereof that is at least 80% identical to one of the nucleic acid sequences shown in Table I as SEQ ID No.1. Preferably, such sequences will be at least about 90% identical, more preferably at least about 95% identical with at least about 99% identical being useful for many screening applications.

Unless otherwise specified, percent sequence identity of two nucleic acids is determined using the algorithm of Karlin and Altschul (1990) PNAS USA 87:2264-2268, modified as in Karlin and Altschul (1993) PNAS USA 90:5873-5877. Such an algorithm is incorporated into the NBLAST and XBLAST programs of Altschul et al (1990) J. Mol. Biol. 215:402-410. BLAST nucleotide searches are performed with the NBLAST program, score=100, word length=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (NBLAST and XBLAST) are used.

More particular examples of such suitable promoter sequences include those having gaps (i.e., contiguous or non-contiguous deletions) in the sequences shown in Table I (SEQ ID No.1) as well as those having nucleotide substitutions, additions, or deletions.

More particular expression vectors in accord with the invention will generally also include sequence 3′ (downstream) from the transcription start site of the DJ-1 genes. Typically, the size or length of such a sequence will be less than about 1000 kb, typically between about 20 base pairs to about 500 base pairs.

Thus in one embodiment, an expression vector of the invention further includes a DJ-1 downstream sequence having about 250 base pairs downstream from the respective human DJ-1 transcription start site, e.g., about 100 base pairs, about 50 base pairs, or about 25 base pairs. Preferred downstream sequences are typically operably linked to the promoter sequence or functional portion thereof. Such linkage can be direct (i.e., direct covalent attachment) or be indirect, such as when the downstream sequence is spaced from the promoter sequence by a spacer element (e.g., less than about 50 base pairs or less than 10 base pairs). In most embodiments, the promoter sequence will be directly linked to the downstream sequence.

Further downstream sequences acceptable for use in the composition or methods of the invention include those that hybridize to any one of the sequences shown in Table I (SEQ ID NO. 1) under high stringency conditions. More preferred downstream sequences are those that are at least 80% identical to one of the nucleic acid sequences shown in Table I as SEQ ID NO.I. Preferably, such sequences will be at least about 90% identical, more preferably at least about 95% identical with at least about 99% identical being useful for many screening applications.

Accordingly, in one embodiment, the invention provides a particular expression vector that includes a human DJ-1 promoter sequence including from about 50 base pairs to 1000 base pairs upstream of the human DJ-1 transcription start site. The promoter sequence further comprising an operably linked DJ-1 downstream sequence, that is about 200 base pairs or less downstream of the human DJ-1 transcription start site. Preferably, the DJ-1 downstream sequence is about 65 base pairs downstream of the human DJ-1 transcription start site.

In addition to the foregoing promoter sequences, the expression vectors will have one or a combination of other components needed to achieve the objects of the invention.

B. Reporter Sequences and Expression Vectors

In particular, most expression vectors will feature a detectable reporter sequence (e.g., luciferase, chloramphenicol transferase, beta-galactosidase) that preferably enables detection of the presence of or the amount of transcription from the vector. As discussed herein, it is an object of the invention to detect compounds that modulate (e.g., decrease) transcription from such vectors. The product of the reporter gene may be virtually any detectable molecule, such as the following biosensors: luciferin (luciferase substrate); aequorin; Fluo-3/acetoxymethyl (esterase substrate); FDG (β-gal substrate); or CCF2, which is a β-lactamase substrate. See generally, J. E. Gonzalez and P. A. Negulescu, Curr. Opin. Biotechnol. 9, 624 (1998). In most embodiments, it will be useful to have a detectable reporter sequence (gene) that encodes a fluorescent, phosphorescent, luminescent, or chemiluminescent protein whose expression can be distinguished from that of other cell components via conventional methods. In some embodiments however, it may be useful to employ reporter sequences that encode proteins detectable by calorimetric methods.

Preferably, the amino acid sequence encoded by the detectable reporter sequence is directly detectable by the assay of the invention. Examples of such suitable sequences include those encoding luciferase, green fluorescent protein (GFP), red fluorescent protein (RFP); as well as fluorescent fragments and derivatives thereof. See e.g., U.S. Pat. Nos. 6,146,826; 5,741,668; 5,804,387; 6,723,537 and 6,391,630.

In embodiments in which luciferase is the detectable amino acid sequence of choice, the enzyme is preferably derived from a bacterium or an insect such as the American firefly (Photinus pyralis) or Renilla.

Examples of suitable luciferase, GFP and RFP fragments have been disclosed in the U.S. Pat. Nos. 6,146,826; 5,741,668; 5,804,387; 6,723,537; 6,391,630; and references cited therein.

More particular expression vectors for use with the invention will include a detectable reporter sequence the encodes what is referred to herein as a “derivative” of the luciferase enzyme i.e., a luciferase that has reduced intracellular stability compared with a naturally-occurring luciferase. Such luciferase derivatives are well-known in the field and include commercially available enzyme derivatives. Preferred luciferase derivatives generally include a genetic mutation that provides to the enzyme one or more of the following characteristics: loss of a mammalian transcription factor binding site, optimization of codon usage, addition of a degradation sequence (e.g., at least one of PEST, ARE (AU-rich element), and CL1 element). By the term “PEST” is meant the forty-amino acid sequence isolated from the C-terminal of mouse ornithine carboxylase. See Li, X. (1998) J. Biol. Chem. 273: 34970. By “CL1” is meant a reported degradation signal. See Gilon, T. et al. (1998) EMBO J. 17: 2759. By “ARE” is meant a disclosed AU-rich element. Fan, X. C. et al. (1997) Genes Dev. 11: 2557.

More preferred luciferase derivatives and vectors encoding the same are commercially available from Promega Corporation (Madison, Wis.). See Promega Technical Manual 242 entitled Rapid Response™ Vectors (December, 2003 version) pp. 1-20 (hereinafter “Promega Technical Manual”). The following Promega vectors are preferred for many invention embodiments: pGL3(R2.1); pGL3(R2.2); phRG(R2.1); and phRG(R2.2). Each of these specific vectors can be conventionally manipulated to include a DJ-1 promoter sequence (including functional fragments) that is disclosed herein.

As should be apparent, the invention is compatible with a broad spectrum of expression vector constructs. In addition to the aforementioned DJ-1 promoter sequences (including functional fragments) and detectable reporter sequences, preferred expression vectors will desirably include additional elements that, for instance, support replication in a microbial host. In this embodiment, the expression vector will include a suitable origin of replication recognized by the intended microbial host and optionally, a promoter, which will function in the host and a phenotypic selection gene such as a gene encoding proteins conferring antibiotic resistance or supplying an autotrophic requirement. Similar constructs will be manufactured for other hosts. E. coli is typically transformed using pBR322. See Bolivar et al., Gene 2, 95 (1977). pBR322 contains genes for ampicillin and tetracycline resistance and thus provides easy means for identifying transformed cells.

More particular expression vectors in accord with the invention include at least one of the following components: 1) SV40 late poly(A) site; 2) ColE1-derived origin of replication; and 3) β-lactamase or functional fragment thereof. In embodiments in which more than one of the foregoing components is present, each will be operably linked to the nucleic acid encoding the detectable reporter sequence. Further specific vectors will further include an f1 origin of replication and an upstream synthetic poly(A) region operably linked to the reporter sequence. Preferred sequences for these elements have been reported. See the Promega Technical Manual, for instance.

Nearly any suitable phenotype selection gene (also known as a drug resistance gene) or a functional fragment thereof can be encoded by the expression vector. Examples of suitable genes include, but are not limited to, neomycin, hypoxanthine phosphoribosyl transferase, puromycin, dihydrooratase, glutamine synthetase, histidine D, carbamyl phosphate synthase, dihydrofolate reductase, multidrug resistance 1 gene, aspartate transcarbamylase, xanthine-guanine phosphoribosyl transferase, adenosine deaminase; or a functional fragment thereof.

In a particular invention embodiment suitable for many invention applications, the expression vector will include at least one of the following; in other embodiments, the expression vector will include all of the following components operably linked in sequence:
1) a polynucleotide encoding a first resistance gene (e.g., ampicillin) or functional fragment thereof,
2) an f1 origin sequence,
3) an upstream synthetic poly(A) region,
4) the DJ-1 promoter sequence (or functional fragment thereof) covalently linked to a corresponding downstream sequence as provided herein,
5) a polynucleotide sequence encoding a luciferase derivative,
6) an SV40 late poly (A) signal; and
7) a polynucleotide encoding a second resistance gene (e.g., neomycin) that is different from the first; or functional fragment thereof.

In a more specific embodiment, expression vector components (1)-(3) and (5)-(7) are provided by vectors disclosed by the Promega Technical Manual. Component (4) is preferably provided by any one of the DJ-1 promoter sequences provided herein which sequence is preferably operably linked to a corresponding downstream sequence, also as provided herein. Although the nucleotide length of component (4) of the expression vector will vary depending, for instance, on intended use, in most cases the length will be less than about 5000 base pairs, preferably less then about 4000 or 3000 base pairs, more preferably between from about 500 to about 2500 base pairs.

Thus in particular invention embodiments, there is provided: 1) a first expression vector that includes expression vector components (1)-(3) and (5)-(7) and a DJ-1 promoter sequence having of about 1000 base pairs and about 65 base pairs of downstream sequence.

Making Cells for Use in Screens

As discussed, the invention is flexible A wide variety of cells and cell lines may be transformed by one or a combination of the expression vectors provided herein. Suitable cells and cell lines are generally eukaryotic and are capable of being transformed by the expression vector. A number of types of cells may act as suitable host cells for the expression vector. Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

In most cases it will be useful to transfect or transform cells that are known or suspected of having cell factors that can, under appropriate conditions, modulate at least one of the DJ-1 promoter sequences provided herein. It is thus an object of the invention to screen candidate compounds that have the potential to modulate (increase or decrease) the activity of such cell factors which modulation is detectable by the reporter sequence.

More particular examples of cells that are believed to harbor cell factors that can interact with one or more of the DJ-1 promoter sequences include those obtained directly from (e.g., a primary cell culture) or derived from the following specific tissues: breast, prostate, testis, neurons, glia, colon, pancreas, stomach, esophagus, astrocytes, lung, lymph, skin; as well as neoplastic or immortalized cell lines obtained from such tissues. A wide variety of such cells and cell lines are readily obtained from the American Type Culture Collection (ATCC, Manassas, Va. (USA)).

Virtually any cell line can be used in the methods of the invention. Particularly useful are cancer cell lines derived from breast, lung, and prostate that overexpress DJ-1. The following Tables II-VIII provide illustrative cells for use with the invention that can be obtained from the ATCC. These cell lines are merely exemplary, and in no way limit the invention. In particular, Table II provides exemplary breast-derived cell lines; Table III provides exemplary lung derived cell lines, Table IV provides exemplary cervix-derived cell lines; Table V provides exemplary prostate-derived cell lines; Table VI provides exemplary tumor cell lines; Table VII provides exemplary testis-derived cell lines; and Table VIII provides non-tumor, neuronal-like cells, tumor-derived neuronal-like cells, glioblastoma cells, medulloblastome-derived cells; retinoblastoma-derived cells; and neuroendocrine tissue.

TABLE II

| ATCC No. | Species | Name | Tissue Source |
|---|---|---|---|
| CRL-7365 | human | Hs 605.T | mammary gland; breast |
| CRL-7368 | human | Hs 606 | mammary gland; breast |
| HTB-126 | human | Hs 578T | mammary gland; breast |
| CRL-2320 | human | HCC1008 | mammary gland; breast |
| CRL-2338 | human | HCC1954 | mammary gland; breast |
| CRL-7345 | human | Hs 574.T | mammary gland; breast |
| CRL-2314 | human | HCC38 | mammary gland; breast |
| CRL-2321 | human | HCC1143 | mammary gland; breast |
| CRL-2322 | human | HCC1187 | mammary gland; breast |
| CRL-2324 | human | HCC1395 | mammary gland; breast |
| CRL-2331 | human | HCC1599 | mammary gland; breast |
| CRL-2336 | human | HCC1937 | mammary gland; breast |
| CRL-2340 | human | HCC2157 | mammary gland; breast |
| CRL-2343 | human | HCC2218 | mammary gland; breast |
| CRL-7482 | human | Hs 742.T | mammary gland; breast |
| CRL-7222* | human | Hs 274.T | mammary |
| CRL-7226* | human | Hs 280.T | mammary |
| CRL-7227* | human | Hs 281.T | mammary |
| CRL-7245* | human | Hs 343.T | mammary |
| CRL-7253* | human | Hs 362.T | mammary |
| CRL-7477* | human | Hs 739.T | mammary |
| CRL-7480* | human | Hs 741.T | mammary |
| CRL-7145* | human | Hs 190.T | mammary gland |
| CRL-7236* | human | Hs 319.T | mammary gland |
| CRL-7242* | human | Hs 329.T | mammary gland |
| CRL-7246* | human | Hs 344.T | mammary gland |
| CRL-7248* | human | Hs 350.T | mammary gland |
| CRL-7256* | human | Hs 371.T | mammary gland |
| CRL-7486* | human | Hs 748.T | mammary gland |
| CRL-7574* | human | Hs 841.T | mammary gland |
| CRL-7583* | human | Hs 849.T | mammary gland |
| CRL-7584* | human | Hs 851.T | mammary gland |
| CRL-7596* | human | Hs 861.T | mammary gland |
| CRL-7652* | human | Hs 905.T | mammary gland |
| CRL-7813* | human | Hs 479.T | mammary gland |
| CRL-7316* | human | Hs 540.T | mammary gland |
| CRL-7336* | human | Hs 566(B).T | mammary gland |
| CRL-7365* | human | Hs 605.T | mammary gland |
| CRL-7368* | human | Hs 606 | mammary gland |
| HTB-19 | human | BT-20 | mammary gland |
| CRL-1897 | human | UACC-812 | mammary gland |
| CRL-2338 | human | HCC1954 | mammary gland |
| CRL-7345* | human | Hs 574.T | mammary gland |
| HTB-121 | human | BT-483 | mammary gland |
| HTB-122 | human | BT-549 | mammary gland |
| HTB-123 | human | DU4475 | mammary gland |
| HTB-126 | human | Hs 578T | mammary gland |
| HTB-20 | human | BT-474 | mammary gland |
| CRL-1902 | human | UACC-893 | mammary gland |
| CRL-2315 | human | HCC70 | mammary gland |
| CRL-2316 | human | HCC202 | mammary gland |
| CRL-2326 | human | HCC1419 | mammary gland |
| CRL-2329 | human | HCC1500 | mammary gland |
| CRL-2330 | human | HCC1569 | mammary gland |
| HTB-27 | human | MDA-MB-361 | mammary gland |
| CRL-2327 | human | HCC1428 | mammary gland |
| CRL-2351 | human | AU565 | mammary gland |
| HTB-128 | human | MDA-MB-415 | mammary gland |
| HTB-130 | human | MDA-MB-436 | mammary gland |
| HTB-132 | human | MDA-MB-468 | mammary gland |
| HTB-21 | human | CAMA-1 | mammary gland |
| HTB-22 | human | MCF7 | mammary gland |
| HTB-26 | human | MDA-MB-231 | mammary gland |
| HTB-30 | human | SK-BR-3 | mammary gland |
| Hs 742.T | human | CRL-7482* | mammary gland |
| HT 762.T | human | CRL-7789* | Breast, nipple |
| HCC1008 | human | CRL-2320 | mammary gland |
| MDA-MB-435S | human | HTB-129 | mammary gland |
| T-47D | human | HTB-133 | mammary gland |
| MDA-MB-134-VI | human | HTB-23 | mammary gland |
| MDA-MB-175-VII | human | HTB-25 | mammary gland |
| ZR-75-1 | human | CRL-1500 | mammary gland |
| ZR-75-30 | human | CRL-1504 | mammary gland |

TABLE III

| Species | Cell Line Name | ATCC No. | |
|---|---|---|---|
| human | NCI-H1581 | CRL-5878 | Adenocarcinoma, lung |
| human | NCI-H23 | CRL-5800 | Adenocarcinoma, lung |
| human | NCI-H522 | CRL-5810 | Adenocarcinoma, lung |
| human | NCI-H1435 | CRL-5870 | Adenocarcinoma, lung |
| human | NCI-H1563 | CRL-5875 | Adenocarcinoma, lung |
| human | NCI-H1651 | CRL-5884 | Adenocarcinoma, lung |
| human | NCI-H1734 | CRL-5891 | Adenocarcinoma, lung |
| human | NCI-H1793 | CRL-5896 | Adenocarcinoma, lung |
| human | NCI-H1838 | CRL-5899 | Adenocarcinoma, lung |
| human | NCI-H1975 | CRL-5908 | Adenocarcinoma, lung |
| human | NCI-H2073 | CRL-5918 | Adenocarcinoma, lung |
| human | NCI-H2085 | CRL-5921 | Adenocarcinoma, lung |
| human | NCI-H2228 | CRL-5935 | Adenocarcinoma, lung |
| human | NCI-H2342 | CRL-5941 | Adenocarcinoma, lung |
| human | NCI-H2347 | CRL-5942 | Adenocarcinoma, lung |
| human | NCI-H2135 | CRL-5926 | Cancer, lung |
| human | NCI-H2172 | CRL-5930 | Cancer, lung |
| human | NCI-H2444 | CRL-5945 | Cancer, lung |
| human | NCI-H835 | CRL-5843 | Carcinoid, lung |
| human | DMS 79 | CRL-2049 | Carcinoma, lung |
| human | DMS 53 | CRL-2062 | Carcinoma, lung |
| human | DMS 114 | CRL-2066 | Carcinoma, lung |
| human | SW 1271 | CRL-2177 | Carcinoma, lung |
| human | NCI-H2227 | CRL-5934 | Carcinoma, lung |
| human | NCI-H1963 | CRL-5982 | Carcinoma, lung |
| human | SHP-77 | CRL-2195 | Carcinoma, lung |
| human | NCI-H1648 | CRL-5882 | Adenocarcinoma, lung |
| human | NCI-H1819 | CRL-5897 | Adenocarcinoma, lung |
| human | NCI-H2009 | CRL-5911 | Adenocarcinoma, lung |
| human | NCI-H1355 | CRL-5865 | Adenocarcinoma, lung |
| human | NCI-H1792 | CRL-5895 | Adenocarcinoma, lung |
| human | NCI-H676B | HTB-179 | Adenocarcinoma, lung |
| human | Calu-3 | HTB-55 | Adenocarcinoma, lung |
| human | NCI-H1573 | CRL-5877 | Adenocarcinoma, lung |
| human | NCI-H1650 | CRL-5883 | Adenocarcinoma, lung |
| human | NCI-H1666 | CRL-5885 | Adenocarcinoma, lung |
| human | NCI-H1781 | CRL-5894 | Adenocarcinoma, lung |
| human | NCI-H2405 | CRL-5944 | Adenocarcinoma, lung |
| human | NCI-H1755 | CRL-5892 | Adenocarcinoma, lung |
| human | NCI-H838 | CRL-5844 | Adenocarcinoma, lung |
| human | NCI-H920 | CRL-5850 | Adenocarcinoma, lung |
| human | NCI-H1568 | CRL-5876 | Adenocarcinoma, lung |
| human | NCI-H1623 | CRL-5881 | Adenocarcinoma, lung |
| human | NCI-H1693 | CRL-5887 | Adenocarcinoma, lung |
| human | NCI-H1993 | CRL-5909 | Adenocarcinoma, lung |
| human | NCI-H2023 | CRL-5912 | Adenocarcinoma, lung |
| human | NCI-H2030 | CRL-5914 | Adenocarcinoma, lung |
| human | NCI-H2087 | CRL-5922 | Adenocarcinoma, lung |
| human | NCI-H2291 | CRL-5939 | Adenocarcinoma, lung |
| human | NCI-H969 | CRL-5852 | Adenocarcinoma, lung |
| human | NCI-H1437 | CRL-5872 | Adenocarcinoma, lung |
| human | NCI-H2122 | CRL-5985 | Adenocarcinoma, lung |
| human | NCI-H1944 | CRL-5907 | Adenocarcinoma, lung |
| human | NCI-H1404 | CRL-5819 | Adenocarcinoma, lung |
| human | NCI-H820 | HTB-181 | Adenocarcinoma, lung |
| human | NCI-H441 | HTB-174 | Adenocarcinoma, lung |
| human | NCI-H650 | CRL-5835 | Carcinoma, lung |
| human | ChaGo-K-1 | HTB-168 | Carcinoma, lung |
| human | NCI-H1694 | CRL-5888 | Carcinoma, lung |
| human | NCI-H711 | CRL-5836 | Carcinoma, lung |
| human | NCI-H719 | CRL-5837 | Carcinoma, lung |
| human | NCI-H1092 | CRL-5855 | Carcinoma, lung |
| human | NCI-H1284 | CRL-5861 | Carcinoma, lung |
| human | NCI-H250 | CRL-5828 | Carcinoma, lung |
| human | NCI-H889 | CRL-5817 | Carcinoma, lung |
| human | NCI-H740 | CRL-5840 | Carcinoma, lung |
| human | NCI-H748 | CRL-5841 | Carcinoma, lung |
| human | NCI-H1105 | CRL-5856 | Carcinoma, lung |
| human | NCI-H1436 | CRL-5871 | Carcinoma, lung |
| human | NCI-H1876 | CRL-5902 | Carcinoma, lung |
| human | NCI-H1930 | CRL-5906 | Carcinoma, lung |
| human | NCI-H1994 | CRL-5910 | Carcinoma, lung |
| human | NCI-H2059 | CRL-5916 | Carcinoma, lung |
| human | NCI-H187 | CRL-5804 | Carcinoma, lung |
| human | NCI-H378 | CRL-5808 | Carcinoma, lung |
| human | NCI-H60 | CRL-5821 | Carcinoma, lung |
| human | NCI-H220 | CRL-5825 | Carcinoma, lung |
| human | NCI-H847 | CRL-5846 | Carcinoma, lung |
| human | NCI-H865 | CRL-5849 | Carcinoma, lung |
| human | NCI-H1304 | CRL-5862 | Carcinoma, lung |
| human | NCI-H2081 | CRL-5920 | Carcinoma, lung |
| human | NCI-H774 | CRL-5842 | Carcinoma, lung |
| human | Tera-1 | HTB-105 | Carcinoma, lung |
| human | Tera-2 | HTB-106 | Carcinoma, lung |
| human | Calu-1 | HTB-54 | Carcinoma, lung |
| human | Hs 284.Pe | CRL-7228 | Carcinoma, lung |
| human | NCI-H460 | HTB-177 | Carcinoma, lung |
| human | NCI-H661 | HTB-183 | Carcinoma, lung |
| human | NCI-H1299 | CRL-5803 | Carcinoma, lung |
| human | NCI-H1155 | CRL-5818 | Carcinoma, lung |
| human | NCI-H2106 | CRL-5923 | Carcinoma, lung |
| human | NCI-H1915 | CRL-5904 | Carcinoma, lung |
| human | NCI-H647 | CRL-5834 | Carcinoma, lung |
| human | NCI-H1770 | CRL-5893 | Carcinoma, lung |
| human | NCI-H1385 | CRL-5867 | Carcinoma, lung |
| human | NCI-H211 | CRL-5824 | Carcinoma, lung |
| human | NCI-H1238 | CRL-5859 | Carcinoma, lung |
| human | NCI-H1618 | CRL-5879 | Carcinoma, lung |
| human | NCI-H1882 | CRL-5903 | Carcinoma, lung |
| human | NCI-H2195 | CRL-5931 | Carcinoma, lung |
| human | NCI-H2196 | CRL-5932 | Carcinoma, lung |
| human | NCI-H2107 | CRL-5983 | Carcinoma, lung |
| human | NCI-H2108 | CRL-5984 | Carcinoma, lung |
| human | NCI-H209 | HTB-172 | Carcinoma, lung |
| human | NCI-H146 | HTB-173 | Carcinoma, lung |
| human | NCI-H345 | HTB-180 | Carcinoma, lung |
| human | NCI-H1870 | CRL-5901 | Carcinoma, lung |
| human | NCI-H1341 | CRL-5864 | Carcinoma, lung |
| human | DMS 153 | CRL-2064 | Carcinoma, lung |
| human | NCI-H735 | CRL-5978 | Carcinoma, lung |
| human | NCI-H1184 | CRL-5858 | Carcinoma, lung |
| human | NCI-H1926 | CRL-5905 | Carcinoma, lung |
| human | NCI-H2029 | CRL-5913 | Carcinoma, lung |
| human | NCI-H2141 | CRL-5927 | Carcinoma, lung |
| human | NCI-H2198 | CRL-5933 | Carcinoma, lung |
| human | NCI-H2330 | CRL-5940 | Carcinoma, lung |
| human | NCI-H1048 | CRL-5853 | Carcinoma, lung |
| human | NCI-H1522 | CRL-5874 | Carcinoma, lung |
| human | NCI-H2171 | CRL-5929 | Carcinoma, lung |
| human | NCI-H69 | HTB-119 | Carcinoma, lung |
| human | NCI-H128 | HTB-120 | Carcinoma, lung |
| human | NCI-H446 | HTB-171 | Carcinoma, lung |
| human | NCI-H82 | HTB-175 | Carcinoma, lung |
| human | NCI-H1869 | CRL-5900 | Carcinoma, lung |
| human | SK-MES-1 | HTB-58 | Carcinoma, lung |
| human | NCI-H226 | CRL-5826 | Carcinoma, lung |
| human | NCI-H526 | CRL-5811 | Carcinoma, lung |
| human | NCI-H524 | CRL-5831 | Carcinoma, lung |
| human | NCI-H841 | CRL-5845 | Carcinoma, lung |
| human | NCI-H196 | CRL-5823 | Carcinoma, lung |
| human | Hs229.T | CRL-7194* | Carcinoma, lung |
| human | Hs573.T | CRL-7343 | Carcinoma, lung |
| human | UMC-11 | CRL-5975 | Carcinoma, lung |
| human | NCI-H727 | CRL-5815 | Carcinoma, lung |
| human | NCI-H720 | CRL-5838 | Carcinoma, lung |
| human | NCI-N417 | CRL-5809 | Carcinoma, lung |
| human | NCI-H2170 | CRL-5928 | Carcinoma, lung |
| human | NCI-H520 | HTB-182 | Carcinoma, lung |
| human | SW 900 | HTB-59 | lung |

TABLE IV

| Species | Cell Line Name | ATCC No. | Source |
|---|---|---|---|
| human | HeLa 229 | CCL-2.1 | cervix |
| human | HeLa S3 | CCL-2.2 | cervix |
| human | H1HeLa | CRL-1958 | cervix |
| human | Hs 588.T | CRL-7850* | cervix |
| human | C-4 I | CRL-1594 | cervix |
| human | C-4 II | CRL-1595 | cervix |
| human | DoTc2 4510 | CRL-7920* | cervix |
| human | C-33 A | HTB-31 | cervix |
| human | SW756 | CRL-10302 | cervix |
| human | SiHa | HTB-35 | cervix |
| human | HT-3 | HTB-32 | cervix |
| human | MS751 | HTB-34 | cervix |
| human | Ca Ski | CRL-1550 | cervix |
| human | ME-180 | HTB-33 | cervix |

TABLE V

| Species | Cell Line Name | ATCC No. | Source |
|---|---|---|---|
| human | RWPE-1 | CRL-11609 | transfected with Ki-MSV |
| human | RWPE-2 | CRL-11610 | transfected with HPV-18 |
| | | | and Ki-MSV |
| human | PWR-1E | CRL-11611 | immortalized with Ad12- |
| | | | SV40 hybrid virus |
| human | PZ-HPV-7 | CRL-2221 | epithelium; HPV-18 |
| | | | transformed |
| human | 22Rv1 | CRL-2505 | prostate |
| human | PC-3 | CRL-1435 | prostate |
| human | MDA PCa 2b | CRL-2422 | prostate |
| human | DU 145 | HTB-81 | prostate |
| human | LNCaP clone FGC | CRL-1740 | prostate |
| human | NCI-H660 | CRL-5813 | prostate |

TABLE VI

| Species | Cell Line Name | ATCC No. | Description |
|---|---|---|---|
| human | MCF 10A | CRL-10317 | fibrocystic disease |
| human | MCF 10F | CRL-10318 | fibrocystic disease |
| human | MCF-10-2A | CRL-10781 | fibrocystic disease |
| human | MCF-12A | CRL-10782 | |
| human | MCF-12F | CRL-10783 | |
| human | Hs 564(E).Mg | CRL-7329 | |
| human | Hs 565(A).Mg | CRL-7330 | cyst |
| human | Hs 565(D).Mg | CRL-7333 | cyst |
| human | Hs 579.Mg | CRL-7347 | |
| human | Hs 617.Mg | CRL-7379 | |
| human | Hs 873.T | CRL-7610 | abnormal |
| human | Hs 874.T | CRL-7611 | abnormal |
| human | Hs 875.T | CRL-7612 | abnormal |
| human | Hs 877.T | CRL-7613 | abnormal |
| human | Hs 879(B).T | CRL-7615 | |
| human | Hs 880.T | CRL-7616 | abnormal |
| human | Hs 885.T | CRL-7618 | abnormal |
| human | Hs 912.T | CRL-7661 | abnormal |
| human | Hs 938.T | CRL-7688 | abnormal |
| human | SW527 | CRL-7940 | Paget's disease |
| human | 184A1 | CRL-8798 | epithelium; |
| | | | chemically |
| | | | transformed |
| human | 184B5 | CRL-8799 | epithelium; |
| | | | chemically |
| | | | transformed |

TABLE VII

| Species | Cell Line Name | ATCC No. |
|---|---|---|
| human | Hs 1.Tes | CRL-7002 |
| human | Hs 181.Tes | CRL-7131 |

TABLE VIII

| ATCC No. | Species | Name | Tissue Source |
|---|---|---|---|
| CRL-10442 | human | HCN-1A | brain |
| CRL-10742 | human | HCN-2 | brain |
| CCL-127 | human | IMR-32 | brain; neuroblastoma |
| CRL-1718 | human | CCF-STTG1 | brain; astrocytoma |
| CRL-2060 | human | PFSK-1 | brain; cerebellum; |
| | | | malignant primitive |
| | | | neuroectodermal tumor |
| CRL-2137 | human | SK-N-AS | brain; neuroblastoma |
| CRL-2142 | human | SK-N-FI | brain; neuroblastoma |
| CRL-2149 | human | SK-N-DZ | brain; neuroblastoma |
| CRL-2266 | human | SH-SY5Y | brain; neuroblastoma |
| CRL-2267 | human | BE(2)-M17 | brain; neuroblastoma |
| CRL-2268 | human | BE(2)-C | brain; neuroblastoma |
| CRL-2270 | human | MC-IXC | brain; neuroblastoma |
| CRL-2271 | human | SK-N-BE(2) | brain; neuroblastoma |
| CRL-2273 | human | CHP-212 | brain; neuroblastoma |
| CRL-8621 | human | SVGp12 | brain |
| HTB-10 | human | SK-N-MC | brain; |
| | | | neuroepithelioma, |
| | | | metastic site: supra- |
| | | | orbital area |
| HTB-11 | human | SK-N-SH | brain; neuroblastoma, |
| | | | metastic site: bone |
| | | | marrow |
| HTB-12 | human | SW 1088 | brain; astrocytoma |
| HTB-13 | human | SW 1783 | brain; astrocytoma |
| HTB-15 | human | U-118 MG | brain; glioblastoma; |
| | | | astrocytoma |
| CRL-1620 | human | A172 | brain; glioblastoma |
| CRL-1690 | human | T98G | brain; glioblastoma |
| | | | multiforme |
| CRL-2020 | human | DBTRG-05MG | brain; glioblastoma |
| CRL-2365 | human | M059K | brain; malignant |
| | | | glioblastoma; glioma |
| CRL-2366 | human | M059J | brain; malignant |
| | | | glioblastoma; glioma |
| CRL-7773 | human | TE 615.T | brain; |
| | | | ganglioneuroblastoma |
| HTB-138 | human | Hs 683 | brain; glioma |
| HTB-14 | human | U-87 MG | brain; glioblastoma; |
| | | | astrocytoma |
| HTB-148 | human | H4 | brain; neuroglioma |
| HTB-16 | human | U-138 MG | brain; glioblastoma |
| CRL-8805 | human | TE671 | brain; cerebellum; |
| | | subline No. 2 | medulloblastoma |
| HTB-185 | human | D283 Med | brain; cerebellum; |
| | | | medulloblastoma, |
| | | | matastic site: |
| | | | peritoneum |
| HTB-186 | human | Daoy | brain; cerebellum; |
| | | | desmoplastic cerebellar |
| | | | medulloblastoma |
| HTB-187 | human | D341 Med | brain; cerebellum; |
| | | | medulloblastoma |
| HTB-169 | human | WERI-Rb-1 | retinoblastoma; eye; |
| | | | retina |
| HTB-18 | human | Y79 | retinoblastoma; eye; |
| | | | retina |
| CRL-5813 | human | NCI-H660 | lung; carcinoma; small |
| | | | cell lung cancer |
| | | | extrapulmonary origin |
| | | | (prostate), metastic |
| | | | site: lymph node |
| CRL-5893 | human | NCI-H1770 | lung; carcinoma; non- |
| | | | small cell lung cancer; |
| | | | metastic site: lymph |
| | | | node |
| CRL-2139 | human | SK-PN-DW | malignant primitive |
| | | | neuroectodermal |
| | | | tumor; retroperitoneal |
| | | | embryonal tumor |
| CRL-1973 | human | NTERA-2 cl.D1 | malignant pluripotent |
| | | | embryonal carcinoma; |
| | | | testis, metastic site: |
| | | | lung |

A particular example of an appropriate tumor cell line for use with the invention is the human cervical cancer cell line, HeLa, which is described in the Examples. Another particular example of an appropriate tumor cell for use with the invention is the neuroblastoma cell line, SH-SY5Y, which is also described in the Examples.

The cells and cell lines disclosed herein can be transfected with one or more of the expression vectors already described. Typically, just one type of expression vector will be used to transfect the cells. The term “transfection” as used herein means an introduction of a foreign DNA or RNA into a cell by mechanical inoculation, electroporation, infection, particle bombardment, microinjection, or by other known methods. Alternatively, one or a combination of expression vectors can be used to transform the cells and cell lines. The term “transformation” as used herein means a stable incorporation of a foreign DNA or RNA into the cell which results in a permanent, heritable alteration in the cell. A variety of suitable methods are known in the field and have been described. See e.g., Ausubel et al, supra; Sambrook, supra; and the Promega Technical Manual.

In particular invention embodiments, a cell or cell line of choice is manipulated so as to be stably transformed by an expression vector of the invention. However, for some invention embodiments, transient expression of the vector (e.g., for less than about a week, such as one or two days) will be more helpful. Cells and cell lines that are transiently transfected or stably transformed by one or more expression vectors disclosed herein will sometimes be referred to as “recombinant”. By the phrase “recombinant” is meant that the techniques used for making cell or cell line include those generally associated with making and using recombinant nucleic acids (e.g., electroporation, lipofection, use of restriction enzymes, ligases, etc.).

As discussed herein, this also relates to methods for detecting and in some cases analyzing compounds that decrease activity of a DJ-1 promoter (or functional portions thereof). Certain of those compounds can be further selected if needed to identify those with therapeutic capacity to treat or prevent a neoplasm. Preferred detection and analysis methods include both in vitro and in vivo assays to determine the therapeutic capacity of agents to prevent, treat, prolong the onset of, or help alleviate the symptoms of a neoplasm.

### Screening Assays

Typical screening assays according to the invention include at least one of or all of the following steps: (a) contacting a recombinant cell or cell line with at least one candidate compound, where the cell or cell line includes at least a fragment of a DJ-1 gene promoter operably linked to a detectable reporter sequence; (b) incubating the cells under conditions sufficient to express a detectable reporter sequence; and (c) detecting a change in the expression of the reporter sequence (relative to a suitable control) as being indicative of the presence (or absence) of a compound that can modulate a DJ-1 gene promoter. Compounds that decrease the expression of a DJ-1 gene promoter are likely to be useful to prevent, treat, defer the onset of, or reduce symptoms associated with a neoplasm.

A more particular embodiment of the forgoing screening assay features all of the following steps:
1) providing a recombinant cell containing at least an expression vector (e.g., a DJ-1 gene promoter, or fragment thereof, operably linked to a reporter gene) disclosed herein;
2) contacting the cell with a candidate compound under conditions sufficient to allow expression of a detectable reporter sequence (e.g., luciferase, GFP or RFP);
3) detecting the detectable reporter; and
4) comparing the effect of the candidate compound on the expression of the detectable reporter in the contacted cell, where an alteration (e.g., a decrease) in the level of reporter expression (relative to a suitable control) indicates that the candidate compound reduces the activity of the DJ-1 promoter sequences.

In one embodiment of the above assay, the candidate compound is a known compound. The assays described herein can effectively measure the capacity of the candidate compound to modulate the DJ-1 promoter sequence. The recombinant cells can stably express the vector or such expression may be transient. References herein to a “standard in vitro screening assay,” or similar phrases, refers to the above protocol of steps 1) through 4). Suitable cells for conducting the assay are described herein (e.g., Tables II-VIII). Although it is generally preferred that whole cells be used in the assay, some embodiments may be practiced with a lysate of such cells or tissues, or a substantially purified fraction of the lysate may be employed in some cases.

Compositions of the invention are useful for the high-throughput low-cost screening of candidate compounds to identify those that modulate the expression of a DJ-1 polypeptide or nucleic acid molecule. Any number of methods are available for carrying out screening assays to identify new candidate compounds that promote the expression of a DJ-1 gene. In one working example, candidate compounds are added at varying concentrations to the culture medium of cultured cells expressing one of the nucleic acid sequences of the invention. Gene expression is then measured, for example, by microarray analysis, Northern blot analysis (Ausubel et al., supra), reverse transcriptase PCR, or quantitative real-time PCR using any appropriate fragment prepared from the nucleic acid molecule as a hybridization probe. The level of gene expression in the presence of the candidate compound is compared to the level measured in a control culture medium lacking the candidate molecule. A compound that promotes an decrease, in the expression of a DJ-1 gene, a fragment thereof, or a functional equivalent thereof, is considered useful in the invention; such a molecule may be used, for example, as a therapeutic to treat a neoplasm in a human patient.

In another working example, the effect of candidate compounds may be measured at the level of polypeptide production using the same general approach and standard immunological techniques, such as Western blotting or immunoprecipitation with an antibody specific for a polypeptide encoded by a DJ-1 gene. For example, immunoassays may be used to detect or monitor the expression of at least one of the polypeptides of the invention in an organism. Polyclonal or monoclonal antibodies, (produced as described above) that are capable of binding to such a polypeptide may be used in any standard immunoassay format (e.g., ELISA, Western blot, or RIA assay) to measure the level of the polypeptide. In some embodiments, a compound that promotes a decrease in the expression or biological activity of the polypeptide is considered particularly useful. Again, such a molecule may be used, for example, as a therapeutic to delay, ameliorate, or treat a neoplasm in a human patient.

In another working example, a DJ-1 nucleic acid described is expressed as a transcriptional or translational fusion with a detectable reporter, and expressed in an isolated cell (e.g., mammalian or insect cell) under the control of an endogenous promoter. The cell expressing the fusion protein is then contacted with a candidate compound and the expression of the detectable reporter in that cell is compared to the expression of the detectable reporter in an untreated control cell. A candidate compound that alters (e.g., increases or decreases) the expression of the detectable reporter is a compound that is useful for the treatment of a neoplasm. In one embodiment, the candidate compound decreases the expression of a reporter gene fused to a DJ-1 promoter.

Candidate compounds include organic molecules, peptides, peptide mimetics, polypeptides, nucleic acids, and antibodies that bind to a nucleic acid sequence or polypeptide of the invention (e.g., a DJ-1 gene). Candidate compounds that decrease DJ-1 expression are particularly useful in the methods of the invention.

The DJ-1 expression vector and recombinant cells described herein are used in the discovery and development of a therapeutic compound for the treatment of a neoplasm. Optionally, compounds selected using any of the above-described assays are tested for their ability to stabilize the growth, reduce the survival, or inhibit the invasiveness of a neoplastic cell using standard assays known to the skilled artisan.

Neoplastic cell growth is not subject to the same regulatory mechanisms that govern the growth or proliferation of normal cells. Compounds that reduce the growth or proliferation of a neoplasm are useful for the treatment of neoplasms. Methods of assaying cell growth and proliferation are known in the art. See, for example, Kittler et al. (Nature. 432 (7020):1036-40, 2004) and Miyamoto et al. (Nature 416(6883):865-9, 2002). Assays for cell proliferation generally involve the measurement of DNA synthesis during cell replication. In one embodiment, DNA synthesis is detected using labeled DNA precursors, such as ([³H]-Thymidine or 5-bromo-2′-deoxyuridine [BrdU], which are added to cells (or animals) and then the incorporation of these precursors into genomic DNA during the S phase of the cell cycle (replication) is detected (Ruefli-Brasse et al., Science 302(5650):1581-4, 2003; Gu et al., Science 302 (5644):445-9, 2003).

Candidate compounds that reduce the survival of a neoplastic cell are also useful as anti-neoplasm therapeutics. Assays for measuring cell viability are known in the art, and are described, for example, by Crouch et al. (J. Immunol. Meth. 160, 81-8); Kangas et al. (Med. Biol. 62, 338-43, 1984); Lundin et al., (Meth. Enzymol. 133, 27-42, 1986); Petty et al. (Comparison of J. Biolum. Chemilum. 10, 29-34, 1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull J. Heterocyclic Chem. 25, 911, 1988). Assays for cell viability are also available commercially. These assays include but are not limited to CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay (Promega).

Candidate compounds that increase neoplastic cell death (e.g., increase apoptosis) are also useful as anti-neoplasm therapeutics. Assays for measuring cell apoptosis are known to the skilled artisan. Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

Neoplastic cells have a propensity to metastasize, or spread, from their locus of origination to distant points throughout the body. Assays for metastatic potential or invasiveness are known to the skilled artisan. Such assays include in vitro assays for loss of contact inhibition (Kim et al., Proc Natl Acad Sci USA. 101:16251-6, 2004), increased soft agar colony formation in vitro (Zhong et al., Int J. Oncol. 24(6):1573-9, 2004), the Lewis lung carcinoma (3LL) model of pulmonary metastasis (Datta et al., In Vivo, 16:451-7, 2002) and Matrigel-based cell invasion assays (Hagemann et al. Carcinogenesis. 25: 1543-1549, 2004). In vivo screening methods for cell invasiveness are also known in the art, and include, for example, tumorigenicity screening in athymic nude mice. A commonly used in vitro assay to evaluate metastasis is the Matrigel-Based Cell Invasion Assay (BD Bioscience, Franklin Lakes, N.J.).

If desired, candidate compounds selected using any of the screening methods described herein are tested for their efficacy using animal models of neoplasia. In one embodiment, mice are injected with neoplastic human cells. The mice containing the neoplastic cells are then injected (e.g., intraperitoneally) with vehicle (PBS) or candidate compound daily for a period of time to be empirically determined. Mice are then euthanized and the neoplastic tissues are collected and analyzed for DJ-1 mRNA or protein levels using methods described herein. Compounds that decrease DJ-1 mRNA or protein expression relative to control levels are expected to be efficacious for the treatment of a neoplasm in a subject (e.g., a human patient). In another embodiment, the effect of a candidate compound on tumor load is analyzed in mice injected with a human neoplastic cell. The neoplastic cell is allowed to grow to form a mass. The mice are then treated with a candidate compound or vehicle (PBS) daily for a period of time to be empirically determined. Mice are euthanized and the neoplastic tissue is collected. The mass of the neoplastic tissue in mice treated with the selected candidate compounds is compared to the mass of neoplastic tissue present in corresponding control mice.

### Test Compounds and Extracts

The standard in vitro screening assay is flexible and can be used to screen one or a combination of different compounds. Illustrative examples follow and include, but are not limited to chemical libraries. In embodiments in which large scale screening is desirable, the methods of the invention can be used to screen, for instance, publicly available chemical libraries. Such libraries include the following: Chem Bridge DiverSet E (16,320 compounds, ChemBridge Corp. San Diego, Calif.); Bionet 1 (4,800 compounds; Ryan Scientific, Isle of Palms, S.C.); CEREP library (4,800 compounds; CEREP, Richmond, Wash.). Further chemical libraries may be obtained from the U.S. National Cancer Institute such as the Structural Diversity Set, version 2 (2,000 compounds); Mechanistic Diversity Set (900 compounds); Open Collection 1 (90,000 compounds), and Open Collection 2 (10,000 compounds).

The Sigma “LOPAC” collection (The Library of Pharmaceutically Active Compounds (Sigma Chemical Company), which consists of 1280 compounds with known clinical/pharmacological/biochemical activity, is another library that may be used in the compound screening methods described herein. This library is widely used in the pharmaceutical industry for assay validation and optimization. The SPECS compound library, which is composed of 11,000 compounds selected from numerous commercial vendors on the basis of structural diversity, and the Maybridge Hits Kit compound library, which is composed of 9,000 structurally diverse compounds based upon the Maybridge chemical scaffolds, are also useful in the methods of the invention. Another compound library that can be used with the invention is the Prestwick Chemical Library (available from Prestwick Chemical, Inc.; Washington D.C). The Prestwick library has been reported to include compounds with known efficacy in different therapeutic areas.

Another compound library that can be used in accord with the invention can be obtained from the U.S. National Institute of Neurological Disorders and Stroke (NINDS). The NINDS Custom Collection includes 1,040 compounds. The Spectrum library from MicroSource Discovery Systems is a 2000-compound library is the commercial-distribution version of the compound library assembled for a neurodegeneration drug screening consortium sponsored by the NINDS. The Spectrum library includes FDA-approved drugs, other compounds with known bioactivity, and natural products.

Further chemical library collections can be used with the invention including those extracts obtained from various plants, fungi and marine sources available from the U.S. National Cancer Institute.

The known or candidate compound can be employed as a sole active agent or in combination with other agents, including other compounds to be tested. In most instances, in vitro assays are performed with a suitable control assay usually comprising the same test conditions as in the steps above, but without adding the compound to the medium (e.g., an equal volume of sterile water or saline is added instead). In such cases, a candidate compound that represses DJ-1 promoter activity can be identified as exhibiting a desired activity by exhibiting at least about 5% percent less activity relative to the control; more preferably at least about 10% less activity relative to the control assay; and still more preferably at least about 30%, at least about 80%, about 100%, about 150% or about 200% less activity or more relative to the control.

Detection of reporter expression in the assays of the invention is achieved by one or a combination of different conventional methods. In one embodiment, the reporter produces an assay signal that is detected by a fluorometric assay. Typical of such fluorometric assays are those that include use of at least one of a fluorescence microscope, fluorometer, fluorescence microplate reader, fluorescence activated cell sorter or flow cytometer.

As will be apparent, it will often be desirable, but not always necessary, to include a suitable control. In one embodiment, the assay signal from the reporter sequence is compared to a baseline (control) signal produced by a control assay. This baseline (control) signal is subtracted from the assay signal to produce a corrected signal indicative of the presence (or absence if it is substantially less then the control) of the compound. It will be appreciated that in instances in which the activity of a reporter sequence expressed by a particular recombinant cell or cell line of the invention is well known, use of a control may not be necessary.

### Screening Formats

The compositions and methods of the invention are readily adaptable for use in an automated, semi-automated, or manual screening format. In one embodiment, the method is conducted in a high throughput screening assay. Such an embodiment will often be preferred when the screen is intended to assay compound libraries. General methods for performing such high throughput screens (HTS) have been reported, for instance, by the Institute of Chemistry and Cell Biology (ICCB) of Harvard University. General disclosure relating to such screens has been reported, for instance, by J. C Yarrow, et al. (2003) in Combinatorial Chemistry, 6: 79 (disclosing particular chemical libraries, equipment, and screens for performing certain high throughput fluorescence detection strategies). See also Smith, R. A et al. (2004) Comb. Chem. High Throughput Screen. 7: 141; and U.S. Pat. Nos. 6,630,311 and 6,444,992 for additional disclosure relating to performing HTS analysis.

In one embodiment of a screen, preferably an HTS assay, the assay uses at least one of and preferably all of the following components (available from Sigma, St. Louis, Mo.; Perkin Elmer, for instance) (1) microtiter plates, (2) fluorometer, fluorescence microplate reader, flow cytometer and a luminometer. Suitable microtitre plates will include about 5000 recombinant cells or cells lines of the invention in about 50 microliters of medium. One or a combination of suitable candidate compounds is present in the medium at a concentration of between from about 0.1 to about 2000 micomolar, preferably about 10 to 50 micromolar, for instance.

In many HTS formats, the screening of a chemical library is preferred. To handle data output efficiently, it will often be useful to interface with a computing device, preferably interfaced with the fluorometer, fluorescence microplate reader, flow cytometer or luminometer, typically to receive input from the assay and provide output data to a user. Such output data can be stored and optionally manipulated by the computer or outputted to the user in real-time.

Nearly any suitable screening assay of the invention can be scaled to the appropriate format (96 or 384 well) for high throughput screening. One reason this may be suitable in some embodiments is that it has been found that the luciferase activity generated is directly correlated with the number of cells. Due to the use of tumor-derived cell lines as backbone cells in some assay embodiments, certain compounds will reduce cell viability at high doses and affect luciferase readout. To ameliorate such a condition, a CMV-driven β-galactosidase gene was expressed as an internal control for toxicity and cell numbers. Although some care must be taken to ensure that detected agents do not regulate the CMV promoter. The HeLa cell based assay system described below, for instance, is useful in the identification of compounds that transcriptionally repress DJ-1, which is required for the survival of some neoplastic cells. Compounds that decrease DJ-1 expression are likely to be useful for the treatment of a neoplasm, such as breast or lung cancer. In one embodiment, such screening is used at an initial phase. FDA approved medications, novel compounds, or herbal supplements with low side effects may repress DJ-1. If desired, candidate compounds identified according to the screening assays of the invention are validated in assays where the level of the DJ-1 protein is assayed. In another embodiment, candidate compounds identified according to the methods described herein are assayed for the ability to reduce neoplastic cell survival, inhibit neoplastic cell growth, or reduce metastatic potential in a neoplastic cell in standard cell culture assays or in animal models.

It will often be helpful to combine the in vitro screens of the invention (including the HTS assays) with one or more in vivo testing strategies. Such testing typically includes testing a compound exhibiting acceptable activity in one or more in vitro assays in an accepted animal model of a human neoplasm.

Thus in embodiments in which testing one or more candidate compounds includes in vivo testing, the method will further include selecting compounds that reduce the severity of or delay the onset of a neoplasia or neoplastic symptoms in the animal by at least about 10%, 25%, 50%, 75% or more as compared to a control. Such methods can be used, for instance, to confirm activity of any of the candidate compounds disclosed herein.

The following Examples are intended to be illustrative of the scope of the present invention.

### EXAMPLES

### Example 1

### Expression Vector for Detecting Agents that Modulate DJ-1

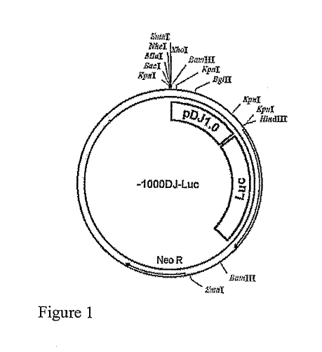

To develop a cell-based assay system where one can identify agents that transcriptionally modulate DJ-1, a plasmid vector (−1000DJ-Luc) was made that expressed a luciferase reporter gene directed by the human DJ-1 promoter containing 1000 base pairs upstream and 65 base pairs downstream sequences (−1000 to +65) of the transcription initiation site (FIG. 1). A 19 base pair sequence containing +46 to +65 of the DJ-1 gene was fused to the luciferase gene to generate the control plasmid (0DJLuc). A neomycin resistance gene was inserted into the vectors as a selection marker.

### Example 2

### SH-SY5Y Cell-Based Assay for Detecting Agents that Modulate DJ-1

Using the vector described in Example 1, human SH-SY5Y neuroblastoma cells were transfected with either −1000DJLuc or 0DJLuc; stably transfected clones were subsequently identified. A plasmid encoding β-galactosidase gene was co-transfected to serve as an internal control for luciferase expression.

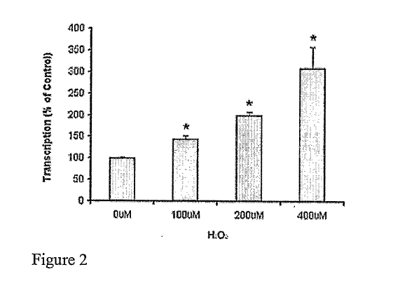

To confirm the stable expression of the reporter genes and to evaluate these cells as devices for drug discovery, stably transfected SH-SY5Y cells were treated with increasing doses of H₂O₂, which has been reported to upregulate DJ-1. As shown in FIG. 2, H₂O₂ activated the human DJ-1 promoter in a dose dependent manner. SH-SY5Y cells stably expressing −1000DJ-Luc and a plasmid encoding CMV-β-galactosidase were treated with the indicated amount of H₂O₂ for 24 hours. Luciferase and β-galactosidase activities were then determined. Because cellular toxicity increases with increasing H₂O₂ dosages, luciferase activity was normalized to β-galactosidase activity, with the value of untreated sample designated as 100. The values shown represent the average of 2 experiments carried out in triplicate. (P<0.05 by Anova). The Luciferase activity in control cell lines expressing 0DJ-Luc was not affected by H₂O₂. To further assess the recombinant cell line as a tool for compound screening, a rational approach to candidate compound selection was undertaken. Candidate compounds capable of modulating the DJ-1 gene promoter based on the analysis of the human DJ-1 promoter were used in the cell-based screening methods of the invention. Such assays are expected to identify compounds capable of reducing or enhancing DJ-1 expression. In one embodiment, candidate compounds that reduce DJ-1 expression are identified as useful for the treatment of a neoplasia.

### Example 3

### Hela Cell-Based Assay for Detecting Agents that Modulate DJ-1

Human cervical carcinoma Hela cell lines were stably transfected with a plasmid (−1000 DJLuc) containing a luciferase reporter gene directed by the promoter sequence of human DJ-1 gene. The promoter sequence contains 1,000 base pairs upstream of the transcriptional initiation site of the DJ-1 gene. In addition, sequences containing a neomycin resistant gene have been inserted in the luciferase plasmid backbone to facilitate selection of transfected cells. As a control, a plasmid (0DJLuc) encoding a luciferase gene lacking the DJ-1 promoter sequence was transfected in parallel in Hela cells. Studies have confirmed that luciferase expression is more than fifty times higher in cells stably expressing −1000DJLuc than in cells stably expressing 0DJ-Luc.

### Example 4

### Real-Time PCR is Used to Monitor DJ-1 mRNA Expression

SH-SY5Y or HeLa cells are treated with a candidate compound. Cells are then harvested for mRNA at various time points after treatment. Total RNA is extracted from the cells treated with candidate compounds at various time points using Trizol reagent (Invitrogen). The total RNA is then purified with the RNAeasy kit (Qiagen). The quality of the RNA is confirmed using agarose (1%) gel electrophoresis. RNA (1 ng) from each sample was then amplified using Roche LightCycler and Qiagen QuantiTect™ SYBR Green RT-PCR kit according to the manufacturer's instructions. This protocol includes: reverse transcription at 50° for 20 minutes, initial activation of Hotstar Taq polymerase at 95° C. for 15 minutes, then 40 cycles of 3-step cycling, including denaturation at 94° C. for 15 seconds; annealing at 58° C. for 20 seconds; extension at 72° C. for 20 sec). A fluorescence detection step is carried out at the end of each cycle. Melting curve analysis (0.1° C. per sec ramping rate, 60-95° C.) was performed with a continuous fluorescence measurement following the 40th cycle. β-actin mRNA was amplified in parallel as an internal control. In one embodiment, candidate compounds that reduce DJ-1 mRNA expression in treated cells relative to untreated control cells are selected as useful for the treatment of a neoplasia.

### Example 5

### DJ-1 Protein Expression is Assayed Using Western Blot

SH-SY5Y or HeLa cells are cultured in vitro. Candidate compounds are administered to the cultures and the cells are then lysed. The protein concentration of each sample is determined using a Protein DC assay (Bio-Rad). Equal amount of proteins (30-40 μg) are resolved by 4-20% Tris-glycine SDS-PAGE and the proteins are transferred onto nitrocellulose membranes, and probed with antibodies against DJ-1 (Stressgen) or β-actin (Santa Cruz). DJ-1 protein is then visualized using standard methods. Candidate compounds that decrease DJ-1 protein levels in a dose dependent fashion relative to untreated control cells are identified as useful in the invention.

### Example 6

### Wild-Type Human DJ-1 Specifically Activates Akt

To investigate a DJ-1 interaction with the Akt/Phosphatidylinositol 3-Kinase (PI3) kinase pathway, the phosphorylation level of Akt was analysed in DJ-1 over-expressing human neuroblastoma SH-SY5Y cells. Akt phosphorylation levels were compared in cells stably expressing mutant DJ-1 (homozygous M26I and heterozygous D149A, which are described by Takahashi et al., Biochem Biophys Res Commun. 320(2):389-97, 2004), myc-his tagged wild-type DJ-1 (Wt), and control cells expressing vector alone. Equivalent amounts of protein samples from each cell type were resolved with SDS-PAGE using duplicating gels, transferred to membranes, and each membrane was separately probed with antibodies specifically recognizing phosphorylated or total Akt. The membranes were then re-probed with an anti-DJ-1 antibody to confirm that similar levels of exogenous DJ-1 expression were present in all of the DJ-1 over-expressing cells.

The phosphatidylinositol 3′ kinase signaling pathway regulates cell growth and survival, and is inhibited by activity of the tumor suppressor PTEN. Abnormally high levels of phosphorylated Akt are associated with neoplasia. As reported herein, increased levels of DJ-1 expression result in the activation of Akt as measured by Akt phosphorylation. The invention provides methods and compositions for identifying compounds that inhibit DJ-1 expression. Compounds that inhibit DJ-1 expression are likely to be useful as chemotherapeutics.

The experiments described in the Examples were carried out using the following methods and materials.

A. Plasmids and Chemicals. DJ-1 promoter sequence from −1000 to +65 relative to the transcriptional initiation site was amplified from pDJ-1(1)luc by PCR with Xho I and Hind III sites using the following primers: DJ-1 F, 5′ GGTGGTCTCGAGGGATCCTTCTAAGCTCATTCAAGA (SEQ ID NO:); DJ-1 R, 5′ GGAGGAAAGCTTTTGGGTACCACTCACCCCA (SEQ ID NO:). The PCR product was then inserted into pGL3 basic vector (Promega), between Xho I and Hind III sites to generate 1000 DJ-Luc vector. For 0DJ-Luc, the sequences from +46 to +65 relative to the transcriptional initiation site were inserted between xhoI and Hind III sites. To facilitate selection, a neomycin resistant gene directed by the SV40 promoter with BamHI and Xho I linker was inserted between the BamHI and SalI sites of pGL3 basic, with the original SalI site abolished after ligation. pON260, which encodes the β-galactosidase gene, was described previously. H₂O₂ was obtained from Sigma.

Fluorometric assays of cell viability and cytotoxicity are performed with the use of a fluorescence microscope, fluorometer, and fluorescence microplate reader or flow cytometer; they offer many advantages over traditional colorimetric and radioactivity-based assays. Also discussed in this section are our unique single-step kits for assessing gram sign and for simultaneously determining gram sign and viability of bacteria.

For information relating to pDJ-1(1)Luc see Taira, T., et al. Gene 263, 285-92 (2001).

B. Cells and Transfection. The human neuroblastoma cell line SH-SY5Y was plated in 6 well dish at 70% confluency and co-transfected with 150 ng of pON260 and 25 fmol of −1000DJ-Luc or 0DJ-Luc per well with Transfectin reagent (Bio-Rad). 24 hours after transfection, cells were re-plated in 10 cm dishes and cultured in medium with 800 μg/ml of Geneticin (G418, Invitrogen). Three days later, cells were selected by culture in medium containing 400 μg/ml of Geneticin. Surviving clones were cultured, expanded, and subjected to analysis.

C. Luciferase and β-galactosidase Assays. Cells were treated with the indicated doses of H₂O₂ for 24 hours before being lysed in reporter lysis buffer (Promega). Luciferase and β-galactosidase activities were determined using assay kits following manufacturer's protocol (Promega).

The following vectors have been disclosed as Genbank/Emb1 accession numbers. pGL3(R2.1)-Basic, AY487821; pGL3(R2.2)-Basic, AY487822; phRG(R2.1)-Basic, AY487823; phRG(R2.2)-Basic, AY487824.

A review of the following specific references will help advance appreciation of the present invention.

### REFERENCES

1. Kim et al., DJ-1, “A novel regulator of the tumor suppressor PTEN,” Cancer Cell. 7(3):263-73 (2005).
2. Hod et al., “Differential control of apoptosis by DJ-1 in prostate benign and cancer cells,” J Cell Biochem. 92(6):1221-33 (2004).
3. Grzmil et al., “Up-regulated expression of the MAT-8 gene in prostate cancer and its siRNA-mediated inhibition of expression induces a decrease in proliferation of human prostate carcinoma cells.” Int J. Oncol. 24(1):97-105 (2004).
4. Yokota et al., “Down regulation of DJ-1 enhances cell death by oxidative stress, ER stress, and proteasome inhibition.” Biochem Biophys Res Commun. 312(4):1342-8 (2003).
5: Srisomsap C et al., “Detection of cathepsin B up-regulation in neoplastic thyroid tissues by proteomic analysis.” Proteomics. 2(6):706-12 (2002).
6. Taira T et al., “Molecular cloning of human and mouse DJ-1 genes and identification of Sp1-dependent activation of the human DJ-1 promoter.” Gene. 263(1-2):285-92 (2001).
7: Nagakubo et al., “DJ-1, a novel oncogene which transforms mouse NIH3T3 cells in cooperation with ras.” Biochem Biophys Res Commun. 231(2):509-13 (1997).
8. Takahashi et al., “DJ-1 Positively Regulates the Androgen Receptor by Impairing the Binding of PIASx to the Receptor,” J Biol Chem 276, 37556-63 (2001).
8. Niki et al., “DJBP: a novel DJ-1-binding protein, negatively regulates the androgen receptor by recruiting histone deacetylase complex, and DJ-1 antagonizes this inhibition by abrogation of this complex,” Mol Cancer Res 1: 247-61 (2003).
10. Le Naour et al., “Proteomics-based identification of RS/DJ-1 as a novel circulating tumor antigen in breast cancer,” Clin Cancer Res 7: 3328-35 (2001).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for detecting a candidate compound that can reduce the growth or survival of a neoplastic cell, the method comprising:
(a) contacting a recombinant cell comprising a DJ-1 expression vector with at least one candidate compound; and
(b) detecting an alteration in expression of DJ-1 in the contacted cell relative to a reference, wherein the method identifies a candidate compound that inhibits the growth or survival of a neoplastic cell.

2. The method of claim 1, wherein the compound decreases DJ-1 transcription or translation or stabilizes the growth or inhibits the invasiveness of the neoplastic cell.

3. (canceled)

4. The method of claim 1, wherein the method further comprises testing the selected compound in a cell growth assay, wherein growth stabilization or growth inhibition identifies the compound as one that stabilizes or inhibits growth of a neoplastic cell.

5. (canceled)

6. The method of claim 1, wherein the method further comprises testing the selected compound in a cell survival assay, wherein a reduction in cell survival identifies the compound as one that reduces the survival of a neoplastic cell.

7. The method of claim 6, wherein the assay detects apoptosis using trypan blue or a TUNEL assay.

8-9. (canceled)

10. The method of claim 6, wherein the method further comprises testing the selected compound in an assay that detects metastatic potential, wherein a compound that reduces metastatic potential is identified as inhibiting the invasiveness of a neoplastic cell.

11. The method of claim 1, wherein the vector comprises between about 500 and 2000 base pairs upstream of a DJ-1 transcription start site operably linked to a detectable reporter.

12. (canceled)

13. The method of claim 11, wherein the upstream sequence is operably linked to a detectable reporter selected from the group consisting of luciferase, green fluorescent protein, chloramphenicol transferase and beta-galactosidase.

14-17. (canceled)

18. The method of claim 1, wherein the method further comprises selecting the candidate compound providing an alteration in DJ-1 expression.

19-24. (canceled)

25. The method of claim 1, wherein the method further comprises testing the compound in a rodent comprising a neoplasm.

26-27. (canceled)

28. The method of claim 25, wherein the compound reduces the growth, proliferation, or severity of the neoplasm by at least 10% when compared to a control.

29. (canceled)

30. A DJ-1 expression vector, the vector comprising a human DJ-1 promoter operably linked to a detectable reporter.

31. (canceled)

32. The DJ-1 expression vector of claim 30, wherein the promoter comprises at least 500 base pairs upstream of a DJ-1 transcription start site operably linked to a detectable reporter.

33. (canceled)

34. The DJ-1 expression vector of claim 30, wherein the promoter comprises from −1000 to +65 relative to the DJ-1 transcriptional initiation site.

35-36. (canceled)

37. A recombinant cell comprising the DJ-1 expression vector of claim 30.

38. The recombinant cell of claim 37, wherein the cell is a human cell derived from a neuronal, breast, lung, testis, or prostate tissue.

39. The recombinant cell of claim 37, wherein the cell is derived from a neuronal, breast, lung, testis, or prostate tissue.

40. The recombinant cell of claim 37, wherein the cell is a neoplastic cell.

41. The recombinant cell of claim 37, wherein the cell is a SH-SY5Y neuroblastoma cell.

42. The recombinant cell of claim 37, wherein the cell is transiently or stably transfected with the expression vector.
